(19) United States
(12) Patent Application Publication
(10) Pub. No.: US 2019/0192455 A1
(43) Pub. Date: Jun. 27, 2019

(54) USE OF CELIPROLOL FOR TREATING KYPHOSCOLIOTIC EHLERS-DANLOS SYNDROME

(71) Applicant: Assistance Publique-Hospitaux de Paris (AP-HP), Paris (FR)

(72) Inventor: Michael Frank, Gambais (FR)

(21) Appl. No.: 16/184,880

(22) Filed: Nov. 8, 2018

(30) Foreign Application Priority Data
Dec. 21, 2017 (EP) 17306888.3

(51) Int. Cl. A61K 31/17; A61P 19/00

(52) U.S. Cl. CPC A61K 31/17; A61B 8/0858; A61P 19/00

(57) ABSTRACT

The present disclosure relates to the use of celiprolol or a pharmaceutically acceptable salt thereof for treating kyphoscoliotic Ehlers-Danlos syndrome.

### CROSS-REFERENCE TO RELATED APPLICATION

This application claims the benefit of European Patent Application No. 17306888.3 filed Dec. 21, 2017, the entire content of which is incorporated herein by reference.

### FIELD

The present disclosure pertains to the field of treatment of an orphan disease, namely the kyphoscoliotic Ehlers-Danlos syndrome (kyphoscoliotic EDS). More specifically, the disclosure pertains to the use of celiprolol or a pharmaceutically acceptable salt thereof for preventing cardiovascular events in kyphoscoliotic EDS patients in need thereof.

### BACKGROUND

The kyphoscoliotic form of the Ehlers-Danlos syndrome (EDS; former EDS type VIA; OMIM#225400) is a rare autosomic recessive connective tissue disorder due to mutations of the lysyl-hydroxylase-1 gene (PLOD1) (Yeowell et al., 2000). The characteristic features of the disease are severe hypotonia at birth, progressive kyphoscoliosis, generalized joint hypermobility and scleral fragility that may lead to rupture of the ocular globe. The presence of three of these clinical criteria is suggestive of the disease and indicates laboratory testing (Beighton et al., 1998; Malfait et al., 2017). Further diagnostic criteria are tissue/skin fragility that may lead to atrophic scars and bruising, a marfanoid habitus, arterial rupture, microcornea, osteopenia and affected sibs (Yeowell et al., 1993). The prevalence of kyphoscoliotic EDS remains unknown. Estimates of incidence are 1:100 000 live-births with a carrier frequency estimated to be 1:150. Pathogenic variants of the PLOD1 gene are characterized by a deficient activity of collagen lysyl-hydroxylase-1 (or procollagen-lysine 2-oxoglutarate 5-dioxygenase 1) leading to an underhydroxylation of collagen lysyl residues and ultimately to an impaired collagen crosslink formation. The diagnosis of kyphoscoliotic EDS can be made by measuring the ratio of urinary lysyl-pyridinoline to hydroxylysyl-pyridinoline (LP/HP) which is expected to be increased in case of a deficient lysyl-hydroxylase-1. Activity of the enzyme can also be tested in cultured skin fibroblasts (Krane et al., 1972). Formal diagnosis is also obtained by genetic testing of the PLOD1 alleles. Kyphoscoliotic EDS has been associated with arterial fragility, but—unlike vascular EDS—arterial ruptures seem to be more prevalent than dissections (a specific type of arterial rupture in which the layers of the vessel separate prior to possible complete failure of the artery wall), and children seem particularly at risk.

Arterial fragility in kyphoscoliotic EDS remains poorly characterized and in absence of effective care, cardiovascular events cause an important morbidity and mortality in kyphoscoliotic EDS patients.

In 2010, Ong et al. reported the results of a clinical trial study for assessing the effects of celiprolol on prevention of cardiovascular events in another rare inherited disease called vascular Ehlers-Danlos syndrome (BBEST study). Vascular EDS is an autosomal dominant disorder resulting from heterozygous mutations in the COL3A1 gene that cause structural defects in the pro-α(1) chain of type III procollagen. The resulting deficiency of structure of type III collagen affects the entire arterial tree, together with the skin and intestine.

Celiprolol is indicated for the management of mild to moderate hypertension and effort-induced angina pectoris. It has a unique pharmacology: it is a selective β1 receptor antagonist, but a β2 receptor partial agonist. It is also a weak α2 receptor antagonist. The BBEST study demonstrated decreased incidence of arterial rupture or dissection in vascular EDS patients taking celiprolol, compared to placebo.

Unlike in kyphoscoliotic EDS, arterial dissections (a specific type of arterial ruptures in which the layers of the vessel separate prior to possible complete failure of the artery wall) are prevalent in vascular EDS. Another difference regarding arterial fragility in these two diseases and is that children having a kyphoscoliotic EDS seem particularly at risk, which is not the case in vascular EDS. Different molecular mechanisms of both diseases may explain these differences in presentation and evolution: indeed, organ fragility in vascular EDS is the direct consequence of a structural weakness of the arterial wall, mainly due to a quantitative type III collagen deficiency (dominant negative effect), whereas kyphoscoliotic EDS is the result of a global enzymatic deficiency (loss of collagen hydroxylation).

The pathophysiology of vascular EDS is hence largely different from that of kyphoscoliotic EDS, so that the results obtained in the BBEST study could not be extrapolated to kyphoscoliotic EDS.

The present disclosure aims at fulfilling the unmet need for prevention of cardiovascular events in kyphoscoliotic EDS patients.

### SUMMARY

The present disclosure pertains to the use of celiprolol or a pharmaceutically acceptable salt thereof for preventing cardiovascular events in kyphoscoliotic EDS patients in need thereof.

The disclosure also pertains to a method for determining if a patient having a kyphoscoliotic Ehlers-Danlos syndrome needs to be treated with celiprolol or a pharmaceutically acceptable salt thereof, comprising measuring the intima-media thickness of an elastic artery (carotid) in said patient, wherein a decreased intima-media thickness indicates that the patient needs to be treated with celiprolol to prevent cardiovascular events.

### DETAILED DESCRIPTION

The present disclosure relates to a novel medical use of celiprolol or a pharmaceutically acceptable salt thereof, namely the treatment of kyphoscoliotic Ehlers-Danlos syndrome.

According to an embodiment, the present disclosure pertains to a method of treating kyphoscoliotic Ehlers-Danlos syndrome, comprising administering celiprolol or a pharmaceutically acceptable salt thereof to a patient in need thereof.

As used herein, the terms “treat”, “treatment” and “treating” refer to any reduction of one or more symptom(s) associated with kyphoscoliotic EDS, such as, for example, a reduction of the occurrence and/or severity of cardiovascular accidents, and/or an increase in survival that results from the administration of celiprolol alone or combined with one or more other therapies.

As used herein, the term “comprise” or “include” is intended to mean that the compositions and methods include the recited elements, but not excluding others. “Consist essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consist of” shall mean excluding more than trace amount of other ingredients and substantial method steps recited. Embodiments defined by each of these transition terms are within the scope of this disclosure.

The term “about” when used before a numerical value indicates that the value may vary within reasonable range, such as ±10%, ±5%, and ±1%. The expression “about x” includes the value “x.”

The singular forms “a” and “the” include plural references unless the context clearly dictates otherwise. Thus, e.g., reference to “the mutation” includes a plurality of mutations.

Celiprolol (brand names Cardem®, Selectol®, Celipres®, Celipro®, Celol®, Cordiax®, Dilanorm®, Edsivo™) is a medication in the class of beta blockers. Its chemical formula is N′-(3-Acetyl-4-(3-((1,1-dimethylethyl)amino)-2-hydroxypropoxy)phenyl)-N, N-diethylurea, its CAS number is 56980-93-9 and Drug Bank number is DB04846.

According to an embodiment of the present disclosure, celiprolol or a pharmaceutically acceptable salt thereof is administered to the patient at a dosage of at least about 50 mg per day, preferably at a dosage ranging from 50 mg to 600 mg per day.

In adult kyphoscoliotic EDS patients, in some embodiments, celiprolol or a pharmaceutically acceptable salt thereof is administered at a dosage superior (or equal) to 100 mg per day, preferably at a dosage ranging from 200 mg to 400 mg of celiprolol or a pharmaceutically acceptable salt thereof per day.

In some embodiments, about 91.25 mg per day of celiprolol or an equivalent amount of a pharmaceutically acceptable salt of celiprolol is administered. In some embodiments, about 182.5 mg (e.g., about 91.25 mg twice daily or about 182.5 mg once daily) to about 365 mg per day (e.g., about 182.5 mg twice daily) of celiprolol or an equivalent amount of a pharmaceutically acceptable salt of celiprolol is administered. An equivalent amount of a pharmaceutically acceptable salt of celiprolol is the weight amount of the salt that provides the stated amount of celiprolol. For example, 200 mg of the HCl salt of celiprolol (celiprolol hydrochloride) provides and is equivalent to 182.5 mg of celiprolol.

In some embodiments, about 100 mg per day (e.g., about 100 mg once daily) of celiprolol hydrochloride is administered. In some embodiments, about 200 mg (e.g., about 200 mg once daily or about 200 mg twice daily) to about 400 mg per day (e.g., about 200 mg twice daily) of celiprolol hydrochloride is administered.

In some embodiments, provided is celiprolol or a pharmaceutically acceptable salt thereof for use in treating kyphoscoliotic Ehlers-Danlos syndrome in a patient, wherein treatment begins with 80 to 110 mg (e.g., about 91.25 mg) daily celiprolol or an equivalent amount of a pharmaceutically acceptable salt of celiprolol and increases to 300 to 440 mg (e.g., about 365 mg) daily celiprolol or an equivalent amount of a pharmaceutically acceptable salt of celiprolol within six months. In some embodiments, provided is a method for treating kyphoscoliotic Ehlers-Danlos syndrome, comprising administering to a patient in need thereof a 80 to 110 mg (e.g., about 91.25 mg) daily dose of celiprolol or an equivalent amount of a pharmaceutically acceptable salt of celiprolol and increasing the daily dose to 300 to 440 mg (e.g., about 365 mg) within six months. In some embodiments, at least a 80 to 110 mg (e.g., about 91.25 mg) daily dose increase is made within two months. In some embodiments, at least a 170 to 210 mg (e.g., about 182.5 mg) daily dose increase is made within four months. In some embodiments, at least a 260 to 310 mg (e.g., about 273.75 mg) daily dose increase is made within six months. In some embodiments, at least a 260 to 310 mg (e.g., about 273.75 mg) daily dose increase is made within four months.

In some embodiments, provided is celiprolol hydrochloride for use in treating kyphoscoliotic Ehlers-Danlos syndrome in a patient, wherein treatment with celiprolol hydrochloride begins with 90 to 110 mg (e.g., about 100 mg) daily and increases to 360 to 440 mg (e.g., about 400 mg) daily within six months. In some embodiments, provided is a method for treating kyphoscoliotic Ehlers-Danlos syndrome, comprising administering to a patient in need thereof a 90 to 110 mg (e.g., about 100 mg) daily dose of celiprolol hydrochloride and increasing the daily dose to 360 to 440 mg (e.g., about 400 mg) within six months. In some embodiments, at least a 90 to 110 mg (e.g., about 100 mg) daily dose increase is made within two months. In some embodiments, at least a 180 to 220 mg (e.g., about 200 mg) daily dose increase is made within four months. In some embodiments, at least a 270-330 mg (e.g., about 300 mg) daily dose increase is made within six months. In some embodiments, at least a 270 to 330 mg (e.g., about 300 mg) daily dose increase is made within four months.

As mentioned above, children having a kyphoscoliotic EDS seem particularly at risk of cardiovascular accidents such as arterial ruptures. According to another aspect of the present disclosure, celiprolol or a pharmaceutically acceptable salt thereof is administered to a child having a kyphoscoliotic EDS. In some embodiments, celiprolol or a pharmaceutically acceptable salt thereof is used in the treatment of a pediatric patient having a kyphoscoliotic EDS. In particular, celiprolol or a pharmaceutically acceptable salt thereof can be administered to teenagers above 14 at a dosage similar to that used for adults. For younger patients, the skilled artisan will adapt the dosage and administration regimen depending on the patient's age, weight and tolerance. For example, dosages ranging from 40 mg once daily to 400 mg per day (e.g., 200 mg twice per day) of celiprolol or a pharmaceutically acceptable salt thereof can be administered.

According to an embodiment of the present disclosure, celiprolol or a pharmaceutically acceptable salt thereof is administered twice daily.

In some embodiments, celiprolol is administered in a pharmaceutical composition comprising celiprolol or a pharmaceutically acceptable salt thereof, and one or more pharmaceutically acceptable excipients. In some embodiments, the pharmaceutical composition is for oral administration. In some embodiments, the pharmaceutical composition is a tablet formulation, such as a film coated tablet. In some embodiments, the pharmaceutical composition is an immediate release formulation, such as an immediate release tablet formulation. In some embodiments, each tablet comprises about 182.5 mg celiprolol or about 200 mg of celiprolol hydrochloride.

In some embodiments, celiprolol is not administered within one hour of a meal. In some embodiments, celiprolol is not administered 1 hour before, or 2 hours after a meal.

In some embodiments, celiprolol is not co-administered with itraconazole, grapefruit juice, orange juice, chlorthalidone, hydrochlorothiazide, theophylline, or rifampicin. In some embodiments, celiprolol is not co-administered with a substrate of MATE1, MATE2-K, BCRP, or P-gp transporter. In some embodiments, celiprolol is not co-administered with calcium channel blockers, such as phenylalkylamine and benzothiazepine, hypotensive agents, or oral antidiabetic (hypoglycemics) drugs. In some embodiments, when co-administered with one or more of the agents, such as itraconazole, the dosage of celiprolol is reduced.

In some embodiments, celiprolol is not co-administered with general anesthesia. In some embodiments, anesthesia is not administered within about 24 hours of the last celiprolol dose. In some embodiments, anesthesia is not administered within about 48 hours of the last celiprolol dose.

In some embodiments, celiprolol is not administered to a patient having one or more of the following conditions: cardiogenic shock, decompensated cardiac failure, sick-sinus syndrome, heart block greater than first degree, severe bradycardia, severe renal impairment with creatinine clearance less than about 15 mL/minute, hypotension, or hypersensitivity to celiprolol.

In some embodiments, when treatment with celiprolol is discontinued, it is discontinued after gradually reducing the dosage over a period of at least one week, such as one to two weeks.

According to an embodiment of the present disclosure, the treatment with celiprolol or a pharmaceutically acceptable salt thereof prevents cardiovascular events. In particular, the treatment with celiprolol or a pharmaceutically acceptable salt thereof prevents arterial rupture, arterial dissection and/or dissecting aneurysm.

According to an embodiment of the present disclosure, the patient is a human. According to an embodiment of the present disclosure, the patient is an adult patient. According to an embodiment of the present disclosure, the patient is pediatric patient.

According to an embodiment of the present disclosure, the patient has a history of arterial dissection or dissecting aneurysm.

According to an embodiment of the present disclosure, the patient has a decreased intima-media thickness of elastic arteries.

According to an embodiment of the present disclosure, the patient is confirmed of having a mutation of the lysyl-hydroxylase-1 gene (PLOD1), such as one or more of composite heterozygous deletion in exon 11 and 12 and deletion of exon 13 and 14 (c.[(1097+1_1098-1)_(1328+1_1329-1)del;(1470+1_1471-1)_(1650+1_1651-1)del]).

According to an embodiment of the present disclosure, the patient has deficient activity of collagen lysyl-hydroxylase-1 (or procollagen-lysine 2-oxoglutarate 5-dioxygenase 1).

According to an embodiment of the present disclosure, the patient has an underhydroxylation of collagen lysyl residues.

According to an embodiment of the present disclosure, the patient has an impaired collagen crosslink formation.

According to an embodiment of the present disclosure, the patient has one or more or three or more of severe hypotonia at birth, progressive kyphoscoliosis, generalized joint hypermobility, and scleral fragility that may lead to rupture of the ocular globe. According to an embodiment of the present disclosure, the patient has one or more of tissue/skin fragility that may lead to atrophic scars or bruising, a marfanoid habitus, arterial rupture, microcomea, osteopenia and affected sibs.

According to an embodiment of the present disclosure, the patient has a history of ocular rupture, vascular event (such as arterial rupture, arterial dissection and/or dissecting aneurysm), kyphoscoliosis and diffuse joint hypermobility. In some embodiments, the rupture or dissection is spontaneous. In some embodiments, the rupture or dissection is recurrent. In some embodiments, the rupture or dissection is a rupture or dissection of a coronary, femoral, intra-thoracic or vertebral artery or a vein.

According to an embodiment of the present disclosure, the patient has one or more symptoms or lesions listed in Table 1 below.

According to an embodiment of the present disclosure, the patient has a family member, such as a sibling, with kyphoscoliotic EDS, such as a sibling with a history of arterial rupture, arterial dissection or dissecting aneurysm.

Another aspect of the present disclosure is a method for determining if a patient having a kyphoscoliotic Ehlers-Danlos syndrome needs to be treated with celiprolol or a pharmaceutically acceptable salt thereof, comprising measuring an intima-media thickness of an elastic artery in said patient. According to this method, a decreased intima-media thickness (such as a thickness of about 500 μm or less, or about 470 μm or less, or about 460 μm or less) indicates that the patient needs to be treated with celiprolol or a pharmaceutically acceptable salt thereof to prevent cardiovascular events.

The above method can be performed for example by measuring the intima-media thickness of a carotid.

As exemplified in the experimental part below, the above method can be performed by measuring the intima-media thickness by high resolution echo tracking.

In certain embodiments, disclosed is
1. Celiprolol or a pharmaceutically acceptable salt thereof, for use in treating kyphoscoliotic Ehlers-Danlos syndrome.
2. Celiprolol or a pharmaceutically acceptable salt thereof, for the use of 1 above, wherein celiprolol or a pharmaceutically acceptable salt thereof is administered to a patient in need thereof at a dosage of at least about 50 mg per day.
3. Celiprolol or a pharmaceutically acceptable salt thereof, for the use of 1 or 2 above, wherein celiprolol or a pharmaceutically acceptable salt thereof is administered to a patient in need thereof at a dosage ranging from about 50 mg to about 600 mg per day.
4. Celiprolol or a pharmaceutically acceptable salt thereof, for the use of any of 1 to 3 above, wherein celiprolol or a pharmaceutically acceptable salt thereof is administered to a patient more than 14 years old at a dosage ranging from about 200 mg to about 600 mg, preferably about 200 mg to about 400 mg of celiprolol or pharmaceutically acceptable salt thereof per day.
5. Celiprolol or a pharmaceutically acceptable salt thereof, for the use of any of 1 to 4 above, wherein celiprolol or a pharmaceutically acceptable salt thereof is administered twice daily.
6. Celiprolol or a pharmaceutically acceptable salt thereof, for the use of any of 1 to 3 above, wherein celiprolol or a pharmaceutically acceptable salt thereof is administered to a patient who is 14 years old or less, at a dosage ranging from about 50 mg to about 400 mg of celiprolol or pharmaceutically acceptable salt thereof per day.
7. Celiprolol or a pharmaceutically acceptable salt thereof, for the use of any of 1 to 6 above, wherein the treatment with celiprolol or a pharmaceutically acceptable salt thereof prevents cardiovascular events.
8. Celiprolol or a pharmaceutically acceptable salt thereof, for the use of any of 1 to 7 above, wherein the treatment with celiprolol or a pharmaceutically acceptable salt thereof prevents arterial rupture, arterial dissection and/or dissecting aneurysm.
9. Celiprolol or a pharmaceutically acceptable salt thereof, for the use of any of 1 to 8 above, wherein celiprolol or a pharmaceutically acceptable salt thereof is administered to a patient who has a history of arterial dissection or dissecting aneurysm.
10. Celiprolol or a pharmaceutically acceptable salt thereof, for the use of any of 1 to 9 above, wherein celiprolol or a pharmaceutically acceptable salt thereof is administered to a patient who has a decreased intima-media thickness of elastic arteries.
11. Celiprolol or a pharmaceutically acceptable salt thereof, for the use of any of 1 to 10 above, wherein celiprolol or a pharmaceutically acceptable salt thereof is administered to a patient who has a sibling with a history of arterial rupture, arterial dissection or dissecting aneurysm.
12. A method of treating kyphoscoliotic Ehlers-Danlos syndrome, comprising administering celiprolol or a pharmaceutically acceptable salt thereof to a patient in need thereof.
13. The method of 12 above, wherein celiprolol or a pharmaceutically acceptable salt thereof is administered to the patient at a dosage of at least 50 mg per day.
14. The method of 12 above, wherein celiprolol or a pharmaceutically acceptable salt thereof is administered to the patient at a dosage ranging from about 50 mg to about 600 mg per day.
15. The method of 12 above, wherein the patient is more than 14 years old and about 200 mg to about 600 mg, preferably 200 mg to 400 mg of celiprolol or a pharmaceutically acceptable salt thereof is administered per day.
16. The method of any one of 12-15 above, wherein celiprolol or a pharmaceutically acceptable salt thereof is administered twice daily.
17. The method of 12 above, wherein the patient is 14 years old or less and about 50 mg to about 400 mg of celiprolol or a pharmaceutically acceptable salt thereof is administered per day.
18. The method of any one of 12-17 above, wherein the treatment with celiprolol or a pharmaceutically acceptable salt thereof prevents cardiovascular events.
19. The method of any one of 12-17 above, wherein the treatment with celiprolol or a pharmaceutically acceptable salt thereof prevents arterial rupture, arterial dissection and/or dissecting aneurysm.
20. The method of any one of 12-19 above, wherein the patient has a history of arterial dissection or dissecting aneurysm.
21. The method of any one of 12-20 above, wherein the patient has a decreased intima-media thickness of elastic arteries.
22. The method of any one of 12-21 above, wherein the patient has a sibling with a history of arterial rupture, arterial dissection or dissecting aneurysm.
23. A method for determining if a patient having a kyphoscoliotic Ehlers-Danlos syndrome needs to be treated with celiprolol or a pharmaceutically acceptable salt thereof, comprising measuring an intima-media thickness of an elastic artery in said patient.
24. The method of 23 above, comprising measuring the intima-media thickness of a carotid.
25. The method of 23 or 24 above, wherein the intima-media thickness is measured by high resolution echo tracking.

Other characteristics of the disclosure will also become apparent in the course of the description which follows of the biological assays which have been performed in the framework of the disclosure and which provide it with the required experimental support, without limiting its scope.

### EXAMPLES

Case Report

A 41 year-old patient was referred to our department for suspicion of connective tissue disorder, following the occurrence of a spontaneous left tibial anterior artery dissection. The dissection was discovered in a context of acute calf pain without identified trauma. Complementary work-up of the arterial tree evidenced a dissecting aneurysm of the celiac trunk, with a flap extending to the superior mesenteric artery (SMA) (as a consequence of an anatomic variation, the SMA originated from the celiac trunk). Two-year prior arterial monitoring had not revealed any arterial defect. Her medical history was relevant for generalized muscle hypotonia at birth, bilateral congenital dislocation of the hip, delayed gross motor development (independent walking at the age of 9 years) and rapidly evolving kyphoscoliosis. Three ocular ruptures occurred at ages 21, 25 and 27 years, respectively. All ruptures occurred following minor trauma. The right eye ruptured twice, the left eye once only. Sympathic ophtalmia complicated the left ocular rupture and resulted in complete loss of vision of the left eye despite adequate treatment. Her family history was remarkable by the death at the age of 9-years of her only and younger sister, by spontaneous abdominal aortic rupture. Her phenotype was similar to her older sister's.

Physical examination evidenced major cutaneous fragility (bruising) and dystrophic scars. Skin elasticity was significantly increased associated with a soft, velvety consistency. She furthermore presented a severe kyphoscoliosis and diffuse joint hypermobility (Beighton score 8/9) with instability, particularly of the left shoulder (recurrent dislocations). Further osteoarticular signs were pes planus and minor pectus excavatum. Echocardiography revealed a minor dysplasia of the mitral valve (anterior leaflet), without significant prolapse. Notably, high resolution echo-tracking recorded an important reduction of the carotid intima-media thickness (453 μm), and a consequent increase of steady circumferential wall stress (75 kPa). Molecular analysis of PLOD1 revealed a composite heterozygous deletion in exon 11 and 12 and deletion of exon 13 and 14 (c.[(1097+1_1098-1)_(1328+1_1329-1)del;(1470+1_1471-1)_(1650+1_1651-1)del]). Urinalysis showed a characteristic increase of the Deoxypyridinoline/Pyridinoline ratio to 10.4, confirming the diagnosis of kyphoscoliotic EDS. The anterior tibial artery aneurysm was managed medically and resulted in minimal sequellae at the site of dissection. The dissecting aneurysm of the celiac trunk also remained stable and a complete regression of the extension of the dissection to the SMA was noted. Two years later, the patient presented with similar pain in the left calf. Doppler ultrasound documented a left tibial anterior dissecting aneurysm. This second clinical event also resolved medically. Celiprolol was initiated and up-titrated to the optimally tolerated dose (400 mg/day) in prevention of further arterial events. The patient remained clinically silent to this day (3 years follow-up) and systematic arterial monitoring did not evidence further silent arterial defects.

Discussion

Kyphoscoliotic EDS is a rare inherited connective tissue disorder, causing a vascular fragility. Indeed, patients with kyphoscoliotic EDS are at risk of spontaneous arterial rupture that may occur at any age, particularly during childhood. This early expression of arterial fragility seems more common than in patients with vascular EDS, which are typically free of symptoms during early childhood (Pepin et al., 2014; Frank et al., 2015). Our case corroborates these findings and illustrates well the potential severity of kyphoscoliotic EDS. Indeed, the proband's family tree suggests that her sister had the same condition and died at the age of 9 years by spontaneous abdominal aortic rupture. By contrast, the proband developed an acute arterial event at the age of 41 years only. The existence of such phenotypic variability within one family has been reported previously for other features, but not in relation with arterial accidents. The patient reported here is to our best knowledge the oldest reported kyphoscoliotic EDS patient, and the first with recurrent spontaneous dissections in medium size arteries without rupture. In regards of this extreme variability, vascular monitoring may be indicated at diagnosis, throughout the patient's life, both in probands and in affected sibs.

Due to the rarity of the disease and in the absence of large characterized patient cohorts, the incidence of arterial accidents in kyphoscoliotic EDS is unknown. However, the predominance of reports of traumatic arterial accidents highlights the exceptional vascular fragility of kyphoscoliotic EDS patients, and makes the occurrence of spontaneous, recurrent arterial accidents all the more likely. In 1989, Wenstrup et al. reviewed the clinical characteristics of n=10 patients with lysyl-hydroxylase deficiency. Ruptures of the femoral, intra-thoracic and vertebral arteries were reported (Wenstrup et al., 1989). Since the molecular characterization of kyphoscoliotic EDS in 2000, only two cases with molecularly proven pathogenic variants and arterial involvement have been reported (Rohrbach et al., 2011; Gok et al., 2012): the first with a coronary artery dissection during a coronarography in a 27 year-old man (reported as spontaneous), and a second with multiple arterial ruptures in medium size arteries starting at the age of 9 years. As for our patient, arterial dissection or dissecting aneurysm seems to be common in medium size arteries beside of the major risk of arterial rupture but they are not yet clearly a part of type VIA EDS definition in the current classification. We also note a fatal case of the superior vena cava rupture after extubation in an 8 year-old girl with molecularly proven disease, underlining again the particular vascular phenotype of the disease (i.e. including venous and arterial lesions) (Working et al., 2015). Published cases of vascular events in kyphoscoliotic EDS patients with sufficient characterization are reported in Table 1. Medicine-related illness and the risk of invasive procedures characterize the majority of reported cases (reporting bias). Spine surgery, likely because of its complexity and the length of the procedures, has a particularly high morbidity. However, in diagnosed patients, using a multidisciplinary approach in the management of the peri-operative period, minimal invasivity during surgery, the patient outcome may be significantly improved (Working et al., 2015).

Since patients with kyphoscoliotic EDS are exposed to acute, possibly life-threatening arterial accidents, it is of critical importance to determine whether it is possible to identify patients that are particularly at risk, and that may benefit from specific monitoring and medical intervention. The relationship between genotype and phenotype has never been evaluated for kyphoscoliotic EDS. The high resolution echotracking findings made in our patient suggest the existence of a detectable predisposition for arterial events. Indeed, a decreased intima-media thickness of elastic arteries was observed in our patient, suggesting that arteries of kyphoscoliotic EDS patients with arterial fragility are exposed to an increased arterial wall stress. Our patient benefited successfully from a treatment by celiprolol, without novel arterial manifestation over a four year follow-up.

### CONCLUSION

Patients with kyphoscoliotic EDS are not only exposed to life-threatening arterial ruptures, but also to spontaneous dissections of medium size arteries. Qualitative assessment of arterial fragility by high resolution echotracking of elastic artery properties may help identify patients/families that are particularly at risk. Patients with increased arterial risk, if not all patients with kyphoscoliotic EDS, may benefit from medical intervention to prevent arterial accidents.

Although the foregoing disclosure has been described in some detail by way of illustration and example for purposes of clarity of understanding, the descriptions and examples should not be construed as limiting the scope of the disclosure. The disclosure of all patent and scientific literature cited herein are expressly incorporated in their entirety by reference.

TABLE 1
Review of type VI Ehlers-Danlos syndromes with vascular events.

| | | | Diagnosis | | | | | Circumstance | |
|---|---|---|---|---|---|---|---|---|---|
| | | | com- | urinary | cultured | molec- | Type | of accident | |
| | | Age | patible | LP/HP | skin | ular | of | (in order of | |
| Patient | Sex | (years) | phenotype | elevation | fibroblasts | analysis | accident | occurrence) | Lesion |
| P1 (Heim | M | 10 | + | ukn | + | ukn | V | I | Internal jugular vein ectasia post- |
| et al., 1992) | | | | | | | | | catheterism |
| P2 (Rohrbach | M | 27 | + | + | ukn | + | A | I | Spontaneous dissection of coronary |
| et al., 2011) | | | | | | | | | arteries during coronary angiography |
| P3 (Esaka | F | ukn | + | + | ukn | ukn | A | T | Right iliac artery rupture at delivery |
| et al., 2009) | | | | | | | | | |
| P4 (Working | F | 8 | + | ukn | ukn | + | V | I | Rupture of the superior vena cava after |
| et al., 2015) | | | | | | | | | extubation |
| P5 (Debnath | M | 20 | + | ukn | ukn | ukn | A | I | Coeliac trunk occlusion after spinal |
| et al., 2007) | | | | | | | | | surgery (mechanism undisclosed) |
| P6 (Bush | M | 32 | + | ukn | ukn | ukn | A/V | S/I/I/S/S | Profundal femoral artery rupture, femoral |
| et al., 2014) | | | | | | | | | false aneurysm (access site), femoral venous |
| | | | | | | | | | rupture, splanchnic artery rupture (30 years), |
| | | | | | | | | | stroke (12 years) (mechanism undisclosed) |
| P7 (Yung | M | 24 | + | ukn | ukn | ukn | A | T/S | Traumatic aortic dissection, popliteal artery |
| et al., 2016) | | | | | | | | | aneurysm (age undisclosed) |
| P8 (Gok | M | 12 | + | + | ukn | + | A | S/S/S | Brachial artery rupture, profundal femoral |
| et al., 2012) | | | | | | | | | artery rupture |
| | | | | | | | | | (11 years) and iliac artery rupture (9 years) |
| P9 (Wenstrup | ukn | ukn | + | ukn | + | ukn | A | ukn | Vertebral artery rupture |
| et al., 1989) | | | | | | | | | |
| P10 (Wenstrup | ukn | ukn | + | ukn | + | ukn | A | S/S | Multiple ruptures of the femoral artery and |
| et al., 1989) | | | | | | | | | repeated spontaneous intrathoracic arterial |
| | | | | | | | | | ruptures |
| P11 (Akpinar | F | 13 | + | ukn | + | ukn | A/V | I/I/I | Abdominal aorta and iliac artery rupture, |
| et al., 2003) | | | | | | | | | common iliac vein rupture during spine |
| | | | | | | | | | surgery |
| P12 (Akpinar | F | 13 | + | ukn | + | ukn | A | I | Intraoperative superior gluteal artery |
| et al., 2003) | | | | | | | | | rupture (spine surgery) |

unk: unknown,
A: arterial;
V: venous;
I: iatrogenic;
S: spontaneous;
T: traumatic

### REFERENCES

Akpinar S, Gogus A, Talu U, Hamzaoglu A, Dikici F. Surgical management of the spinal deformity in Ehlers-Danlos syndrome type VI. Eur Spine J Off Publ Eur Spine Soc Eur Spinal Deform Soc Eur Sect Cerv Spine Res Soc. 2003 Apr. 12(2):135-40. (12)
Beighton P, Paepe A D, Steinmann B, Tsipouras P, Wenstrup R J. Ehlers-Danlos syndromes: Revised nosology, Villefranche, 1997. Am J Med Genet. 1998 Apr. 28; 77(1):31-7.
Boutouyrie P, Germain D P, Fiessinger J N, Laloux B, Perdu J, Laurent S. Increased carotid wall stress in vascular Ehlers-Danlos syndrome. Circulation. 2004 Mar. 30; 109(12):1530-5.
Busch A, Suellner J, Anger F, Meir M, Kickuth R, Lorenz U, et al. Critical care of kyphoscoliotic type Ehlers-Danlos syndrome with recurrent vascular emergencies. Vasa. 2014 May; 43(3):216-21.
Debnath U K, Sharma H, Roberts D, Kumar N, Ahuja S. Coeliac axis thrombosis after surgical correction of spinal deformity in type VI Ehlers-Danlos syndrome: a case report and review of the literature. Spine. 2007 Aug. 15; 32(18): E528-531.
Esaka E J, Golde S H, Stever M R, Thomas R L. A Maternal and Perinatal Mortality in Pregnancy Complicated by the Kyphoscoliotic Form of Ehlers-Danlos Syndrome: Obstet Gynecol. 2009 February; 113(2, Part 2):515-8.
Frank M et al. The type of variants at the COL3A1 gene associates with the phenotype and severity of vascular Ehlers-Danlos syndrome. Eur J Hum Genet. 2015 December; 23(12):1657-64.
Gok E, Goksel O S, Alpagut U, Dayioglu E. Spontaneous Brachial Pseudo-aneurysm in a 12-year-old with Kyphoscoliosis-type Ehlers-Danlos Syndrome. Eur J Vasc Endovasc Surg. November 2012; 44(5):482-4.
Heim P, Raghunath M, Meiss L, Heise U, Myllyla R, Kohlschutter A, et al. Ehlers-Danlos Syndrome Type VI (EDS VI): problems of diagnosis and management. Acta Paediatr. 1998 June; 87(6):708-10.
Krane, S M, Pinnell, S R and R W Erbe. Lysyl-Protocollagen Hydroxylase Deficiency in Fibroblasts from Siblings with Hydroxylysine-Deficient Collagen, Proc. Nat. Acad. Sci. USA. 1972 October; 69(10):2899-903.
Malfait F, Francomano C, Byers P, Belmont J, et al. The 2017 international classification of the Ehlers-Danlos syndromes. Am J Med Genet C Semin Med Genet. 2017 March; 175(1):8-26.
Ong K-T, Perdu J, De Backer J, Bozec E, Collignon P, Emmerich J, et al. Effect of celiprolol on prevention of cardiovascular events in vascular Ehlers-Danlos syndrome: a prospective randomised, open, blinded-endpoints trial. Lancet. 2010 Oct. 30; 376(9751): 1476-84.
Pepin M G, Schwarze U, Rice K M, Liu M, Leistritz D and Byers P H. Survival is affected by mutation type and molecular mechanism in vascular Ehlers-Danlos syndrome (EDS type IV). Genet Med. 2014 December; 16(12):881-8.
Rohrbach M, Vandersteen A, Yis U, Serdaroglu G, Ataman E, Chopra M, et al. Phenotypic variability of the kyphoscoliotic type of Ehlers-Danlos syndrome (EDS VIA): clinical, molecular and biochemical delineation. Orphanet J Rare Dis. 2011; 6:46.
Wenstrup R J, Murad S, Pinnell S R. Ehlers-Danlos syndrome type VI: clinical manifestations of collagen lysyl hydroxylase deficiency. J Pediatr. 1989 September; 115(3):405-9.
Working Z M, Hsiao M, Sanders J C, Bratton S L, D'Astous J L. Spontaneous Fatal Intraoperative Rupture of Great Vessel During Growing Rod Lengthening: Do Children With Ehlers-Danlos Syndrome Require the Availability of Vascular Expertise? A Case Report and Review of the Literature. J Pediatr Orthop. 2017 January; 37(1):e4-e9.
Yeowell H N, Steinmann B. Ehlers-Danlos Syndrome, Kyphoscoliotic Form. In: Pagon R A, Adam M P, Ardinger H H, Wallace S E, Amemiya A, Bean L J, et al, editors. GeneReviews(®) [Internet]. Seattle (Wash.): University of Washington, Seattle; 1993-2017.
Yeowell H N, Walker L C. Mutations in the lysyl hydroxylase 1 gene that result in enzyme deficiency and the clinical phenotype of Ehlers-Danlos syndrome type VI. Mol Genet Metab. October 2000; 71(1 2):212-24.
Yung M Y H, Murray J, Thompson E C. Blunt aortic trauma in a patient with the Ehlers-Danlos syndrome type VI. J Surg Case Rep. 2016 Mar. 7; 2016(3).pii:rjw026.

1. A method of treating kyphoscoliotic Ehlers-Danlos syndrome, comprising administering celiprolol or a pharmaceutically acceptable salt thereof to a patient in need thereof.

2. The method of claim 1, wherein celiprolol or a pharmaceutically acceptable salt thereof is administered to the patient at a dosage of at least 50 mg per day.

3. The method of claim 1, wherein celiprolol or a pharmaceutically acceptable salt thereof is administered to the patient at a dosage ranging from about 50 mg to about 600 mg per day.

4. The method of claim 1, wherein the patient is more than 14 years old and about 200 mg to about 600 mg of celiprolol or a pharmaceutically acceptable salt thereof is administered per day.

5. The method of claim 1, wherein the patient is more than 14 years old and 200 mg to 400 mg of celiprolol or a pharmaceutically acceptable salt thereof is administered per day.

6. The method of any one of claims 1-5, wherein celiprolol or a pharmaceutically acceptable salt thereof is administered twice daily.

7. The method of claim 1, wherein the patient is 14 years old or less and about 50 mg to about 400 mg of celiprolol or a pharmaceutically acceptable salt thereof is administered per day.

8. The method of any one of claims 1-7, wherein the treatment with celiprolol or a pharmaceutically acceptable salt thereof prevents cardiovascular events.

9. The method of any one of claims 1-8, wherein the treatment with celiprolol or a pharmaceutically acceptable salt thereof prevents arterial rupture, arterial dissection and/or dissecting aneurysm.

10. The method of any one of claims 1-9, wherein the patient has a history of arterial dissection or dissecting aneurysm.

11. The method of any one of claims 1-10, wherein the patient has a decreased intima-media thickness of elastic arteries.

12. The method of any one of claims 1-11, wherein the patient has a family member with a history of arterial rupture, arterial dissection or dissecting aneurysm.

13. A method for preventing cardiovascular events in a kyphoscoliotic EDS patient in need thereof, comprising administering celiprolol or a pharmaceutically acceptable salt thereof to the patient.

14. The method of claim 13, wherein celiprolol or a pharmaceutically acceptable salt thereof is administered to the patient at a dosage of at least 50 mg per day.

15. The method of claim 13, wherein celiprolol or a pharmaceutically acceptable salt thereof is administered to the patient at a dosage ranging from about 50 mg to about 600 mg per day.

16. The method of claim 13, wherein the patient is more than 14 years old and about 200 mg to about 600 mg of celiprolol or a pharmaceutically acceptable salt thereof is administered per day.

17. The method of claim 13, wherein the patient is more than 14 years old and 200 mg to 400 mg of celiprolol or a pharmaceutically acceptable salt thereof is administered per day.

18. The method of any one of claims 13-17, wherein celiprolol or a pharmaceutically acceptable salt thereof is administered twice daily.

19. The method of claim 13, wherein the patient is 14 years old or less and about 50 mg to about 400 mg of celiprolol or a pharmaceutically acceptable salt thereof is administered per day.

20. The method of any one of claims 13-19, wherein the treatment with celiprolol or a pharmaceutically acceptable salt thereof prevents arterial rupture, arterial dissection and/or dissecting aneurysm.

21. The method of any one of claims 13-20, wherein the patient has a history of arterial dissection or dissecting aneurysm.

22. The method of any one of claims 13-21, wherein the patient has a decreased intima-media thickness of elastic arteries.

23. The method of any one of claims 13-22, wherein the patient has a family member with a history of arterial rupture, arterial dissection or dissecting aneurysm.

24. A method for determining if a patient having a kyphoscoliotic Ehlers-Danlos syndrome needs to be treated with celiprolol or a pharmaceutically acceptable salt thereof, comprising measuring an intima-media thickness of an elastic artery in said patient.

25. The method of claim 24, comprising measuring the intima-media thickness of a carotid.

26. The method of claim 24 or 25, wherein the intima-media thickness is measured by high resolution echo tracking.
